(19) United States
(12) Patent Application Publication
(10) Pub. No.: US 2025/0104803 A1
(43) Pub. Date: Mar. 27, 2025

(54) METHOD FOR INFORMATION PROCESSING, ELECTRONIC DEVICE, AND STORAGE MEDIUM

(71) Applicant: BEIJING BAIDU NETCOM SCIENCE TECHNOLOGY CO., LTD., Beijing (CN)

(72) Inventors: Kunrui Zhu, Beijing (CN); Lihang Liu, Beijing (CN); Xiaomin Fang, Beijing (CN); Xiaonan Zhang, Beijing (CN); Jingzhou He, Beijing (CN)

(73) Assignee: BEIJING BAIDU NETCOM SCIENCE TECHNOLOGY CO., LTD., Beijing (CN)

(21) Appl. No.: 18/972,078

(22) Filed: Dec. 6, 2024

(30) Foreign Application Priority Data
Jun. 3, 2024 (CN) 202410710910.2

(51) Int. Cl. G16B 15/20; G06F 30/27; G16B 30/00; G16B 40/20

(52) U.S. Cl. CPC G16B 15/20; G06F 30/27; G16B 30/00; G16B 40/20

(57) ABSTRACT

A method for information processing, is performed by an electronic device, and the method includes: obtaining a residue sequence A_{T} that does not carry amino acid information and a first protein backbone structure B_{T} generated by pure noise; and performing iterative denoising on the residue sequence A_{T} and the first protein backbone structure B_{T}; for a t^{th} denoising, obtaining coevolution information of a residue sequence A_{T+1-t}, and obtaining, based on the coevolution information and a first protein backbone structure B_{T+1-t}, a residue sequence A_{T-t} and a first protein backbone structure B_{T-t} after the t^{th} denoising, until the denoising is completed and a target amino acid sequence and a second protein backbone structure are obtained, where t is a positive integer, and 1≤t≤T, and T is a number of denoising times.

### CROSS-REFERENCE TO RELATED APPLICATION

The present application is based upon and claims priority to Chinese Patent Application No. 2024107109102, filed on Jun. 3, 2024, the entire contents of which are incorporated herein by reference.

### TECHNICAL FIELD

The disclosure relates to the technical field of artificial intelligence, in particular to the technical fields of biological computing and protein design, and in particular to a method and an apparatus for information processing, an electronic device and a storage medium.

### BACKGROUND

Proteins, as macromolecular compounds that support an operation of almost all cells, play an important role in different biological functions, such as enzymatic reactions, cell signal transduction, metabolic regulation, and gene expression. The function and three-dimensional structure of protein are largely determined by amino acid types (AATypes) on a protein chain. Proteins with different structures and functions have important application value in many fields such as biomedicine.

With the deepening of protein function research and the development of practical applications, natural proteins can no longer meet the growing needs of mankind, which causes that a method for protein design is of great research value.

### SUMMARY

According to a first aspect of the present disclosure, a method for information processing is provided, which is performed by an electronic device. The method includes: obtaining a residue sequence A_{T} that does not carry amino acid information and a first protein backbone structure B_{T}generated by pure noise; and performing iterative denoising on the residue sequence A_{T} and the first protein backbone structure B_{T}; for a t^{th} denoising, obtaining coevolution information of a residue sequence A_{T+1-t}, and obtaining, based on the coevolution information and a first protein backbone structure B_{T+1-t}, a residue sequence A_{T-t} and a first protein backbone structure B_{T-t} after the t^{th} denoising, until the denoising is completed and a target amino acid sequence and a second protein backbone structure are obtained, in which t is a positive integer, and 1≤t≤T, and T is a number of denoising times.

According to a second aspect of the disclosure, an electronic device is provided. The electronic device includes at least one processor; and a memory communicatively coupled to the at least one processor and storing instructions executable by the at least one processor; in which when the instructions are executed by the at least one processor, the at least one processor is caused to perform the above method for information processing according to the first aspect.

According to another aspect of the disclosure, a non-transitory computer readable storage medium is provided, which stores computer programs/instructions. The computer programs/instructions are used to enable a computer to perform the above method for information processing according to the first aspect.

### BRIEF DESCRIPTION OF THE DRAWINGS

The accompanying drawings are used for a better understanding of the disclosure and do not constitute a limitation of the disclosure.

FIG. 1 is a flowchart of a method for information processing according to an embodiment of the disclosure.

FIG. 2 is a flowchart of another method for information processing according to an embodiment of the disclosure.

FIG. 3 is a diagram of a target protein design network according to an embodiment of the disclosure.

FIG. 4 is a flowchart of a training process of a target protein design network in an method for information processing according to an embodiment of the disclosure.

FIG. 5 is a flowchart of another method for information processing according to an embodiment of the disclosure.

FIG. 6 is a structural diagram of an apparatus for information processing according to an embodiment of the disclosure.

FIG. 7 is a block diagram of an electronic device for implementing the method for information processing according to an embodiment of the disclosure.

### DETAILED DESCRIPTION

Exemplary embodiments of the disclosure are described hereinafter in conjunction with the accompanying drawings, which include various details of the embodiments of the disclosure in order to aid in understanding, and should be considered exemplary only. Accordingly, one of ordinary skill in the art should recognize that various changes and modifications can be made to the embodiments described herein without departing from the scope of the disclosure. Similarly, descriptions of well-known features and structures are omitted from the following description for the sake of clarity and brevity.

The following describes the method and apparatus for information processing, electronic device, and storage medium according to embodiments of the disclosure with reference to the accompanying drawings.

Artificial Intelligence (AI) is a discipline that studies how to use computers to simulate certain thought processes and intelligent behaviors (such as learning, reasoning, thinking, planning, etc.) of human beings. It involves both hardware-level technology and software-level technology. The AI hardware technology generally includes computer vision technology, speech recognition technology, natural language processing technology, as well as learning/deep learning, big data processing technology, knowledge graph technology and other aspects.

Biocomputing is a field where computational problems are solved based on principles and mechanisms from biological systems. Some of the properties and processes of biology are applied to computing systems, so as to improve computing efficiency and performance. The goal of biocomputing is to take inspiration from the biological systems and translate it into new computing methods and technologies to solve complex problems. The biocomputing has a wide range of applications in optimization, mode recognition, data analysis and simulation, and other fields, and it is constantly developing and expanding.

Protein design is to make proteins have a stable folding structure through reasonable amino acid sequence and structural design, so as to achieve specific functions and maintain the structure and function stability under restricted conditions in the living body. The protein design includes artificial modification of proteins and de novo design of proteins. Using computer-aided design methods, new protein molecules with specific functions can be constructed by selecting appropriate amino acids in a protein sequence. This typically includes steps such as target defining, model building, amino acid selecting, protein structure constructing, and evaluation and optimization.

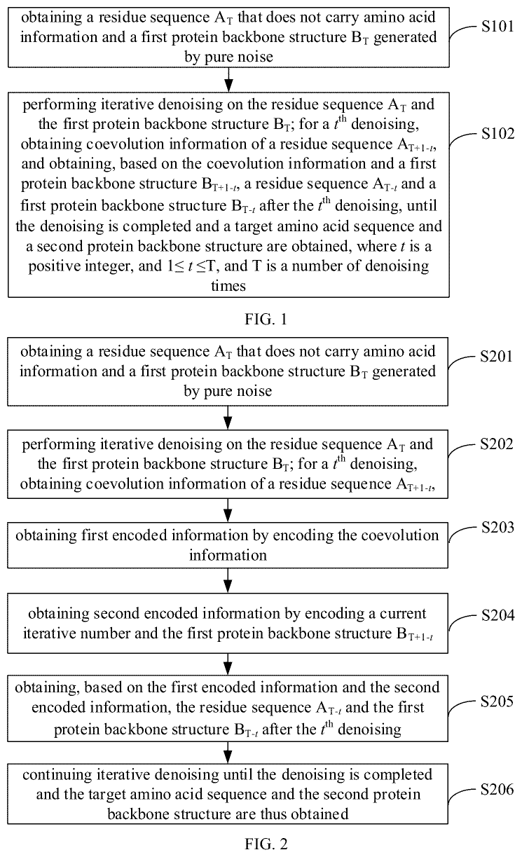

FIG. 1 is a flowchart of a method for information processing according to an embodiment of the disclosure.

As shown in FIG. 1, the method for information processing may include the following steps at S101-S102.

At S101, a residue sequence A_{T} that does not carry amino acid information and a first protein backbone structure B_{T} generated by pure noise are obtained.

It should be noted that the execution body of the method for information processing in the embodiment of the present disclosure may be a hardware device with data processing capabilities and/or software required to drive the hardware device to work. Optionally, the execution body may include a server, a user terminal and other smart device. Optionally, the user terminal includes but is not limited to a mobile phone, a computer, a smart voice interaction device, etc. Optionally, the server includes but is not limited to a network server, an application server, and may also be a server of a distributed system, or a server combined with a blockchain, etc. The embodiments of the present disclosure are not specifically limited.

In some implementations, amino acid information in the amino acid sequence can be removed experimentally to obtain the residue sequence A_{T} that does not carry the amino acid information. The residue sequence A_{T} can also be directly obtained from a database. For example, the residue sequence A_{T} can be obtained from a Protein Data Bank (PDB).

Optionally, noise data may be randomly used to initialize a protein backbone structure, thereby obtaining the first protein backbone structure B_{T} generated by pure noise. For example, the first protein backbone structure B_{T} can be obtained by performing T times of noise addition on any protein backbone structure using the noise.

At S102, iterative denoising on the residue sequence A_{T} and the first protein backbone structure B_{T} is performed; for the t^{th} denoising, coevolution information of a residue sequence A_{T+1-t} is obtained, and a residue sequence A_{T-t} and a first protein backbone structure B_{T-t} after the t^{th} denoising is obtained based on the coevolution information and the first protein backbone structure B_{T+1-t}, until the denoising is completed and a target amino acid sequence and a second protein backbone structure are obtained, where t is a positive integer, and 1≤t≤T, and T is a number of denoising times.

It can be understood that the coevolution information describes coordinated changes between different amino acid sequence sites during the evolution of protein molecules. These changes may include a mutation, a substitution, etc., and these changes are usually not random, but are closely related to the function, structure or other biological properties of the protein. By analyzing the coevolution information, scientists can predict a three-dimensional structure of proteins, understand interactions between proteins and other molecules, and infer changes in the proteins during the evolution

In some implementations, for the t^{th} denoising, the coevolution information of the residue sequence A_{T+1-t} can be determined by performing a multiple sequence alignment (MSA) on the residue sequence A_{T+1-t}. Then, characteristic information of both the coevolution information and the first protein backbone structure B_{T+1-t} are obtained, and the characteristic information is fused to obtain fused characteristic information. The fused characteristic information is further used for structure prediction to achieve residue recovery and protein backbone structure prediction, and the residue sequence A_{T-t} and the first protein backbone structure B_{T-t} after the t^{th} denoising are obtained.

Furthermore, the (t+1)^{th} denoising process is continued based on the residue sequence A_{T-t} and the first protein backbone structure B_{T-t}, until a number of iterations t reaches the number of denoising times T, and the iterative denoising is terminated to obtain the target amino acid sequence (denoted as A₀) and the second protein backbone structure (denoted as B₀).

Optionally, the coevolution information and the first protein backbone structure B_{T+1-t} may be encoded based on a deep neural network to obtain the characteristic information of both the coevolution information and the first protein backbone structure B_{T+1-t}.

According to the method for information processing provided in the embodiment of the present disclosure, the residue sequence A_{T} and the first protein backbone structure B_{T} are obtained, the coevolution information of the residue sequence A_{T} is obtained, iterative denoising is performed based on the coevolution information and the first protein backbone structure B_{T}, to achieve the residue recovery of the amino acid sequence and the prediction of the protein backbone structure, and the target amino acid sequence and the second protein backbone structure are finally obtained. The coevolution information can reveal the interactions between amino acid residues. Combining the coevolution information when denoising helps retain key interactions during the denoising process, thereby improving the accuracy of predicting an amino acid sequence and a protein backbone structure, and enhancing the stability of a protein structure, thereby improving the effects of a protein design.

FIG. 2 is a flowchart of another method for information processing according to an embodiment of the disclosure.

As shown in FIG. 2, the method for information processing may include the following steps at S201-S206.

At S201, a residue sequence A_{T} that does not carry amino acid information and a first protein backbone structure B_{T} generated by pure noise are obtained.

At S202, iterative denoising on the residue sequence A_{T} and the first protein backbone structure B_{T} is performed; for the t^{th} denoising, coevolution information of a residue sequence A_{T+1-t} is obtained.

The relevant contents of steps S201-S202 can be referred to those steps S101-S102 in the above embodiment, which will not be described again here.

At S203, first encoded information is obtained by encoding the coevolution information.

At S204, second encoded information is obtained by encoding a current iterative number and the first protein backbone structure B_{T+1-t}.

In some implementations, the coevolution information and the first protein backbone structure B_{T+1-t} can be encoded with two different deep neural networks respectively to obtain first encoded information and second encoded information.

Optionally, the coevolution information may be encoded as the first encoded information based on an amino acid type (AAType) encoder. The first protein backbone structure B_{T+1-t} can be encoded as the second encoded information based on a structure encoder.

At S205, the residue sequence A_{T-t} and the first protein backbone structure B_{T-t} after the t^{th} denoising are obtained based on the first encoded information and the second encoded information.

In some implementations, the first encoded information and the second encoded information can be fused to obtain fused encoded information, and the residue sequence A_{T+1-t} and the first protein backbone structure B_{T+1-t} are designed respectively based on the fused encoded information, to obtain a structurally accurate and stable denoising result.

Optionally, a residue type design on the residue sequence A_{T+1-t} can be performed based on the fused encoded information to obtain the residue sequence A_{T-t}, and a structure design on the first protein backbone structure B_{T+1-t} can be performed based on the fused encoded information to obtain the first protein backbone structure B_{T-t}.

At S206, iterative denoising is continued to perform until the denoising is completed and the target amino acid sequence and the second protein backbone structure are thus obtained.

The relevant contents of step S206 can be referred to the step S202 in the above embodiment, which will not be described again here.

In some implementations, the iterative denoising on the residue sequence A_{T} and the first protein backbone structure B_{T} can also be performed based on a pre-trained target protein design network, which improves the efficiency of sequence recovery and structure prediction in the protein design.

That is to say, the residue sequence A_{T} and the first protein backbone structure B_{T} are inputted into the pre-trained target protein design network, and the second protein backbone structure and the target amino acid sequence are outputted by performing iterative denoising on the residue sequence A_{T} and the first protein backbone structure B_{T} via the target protein design network.

In some implementations, a protein language model can be introduced into the target protein design network to obtain the coevolution information of the residue sequence, and denoising (i.e., noise reduction processing) can be performed based on the coevolution information, which can improve a matching degree between the amino acid sequence and the protein structure and improve the denoising effect of the sequence.

That is to say, for the t^{th} denoising, the protein language model in the target protein design network extracts the coevolution information of the residue sequence A_{T+1-t}. Then, the residue sequence A_{T-t} and the first protein backbone structure B_{T-t} after the t^{th} denoising are obtained based on the coevolution information and the first protein backbone structure B_{T+1-t}.

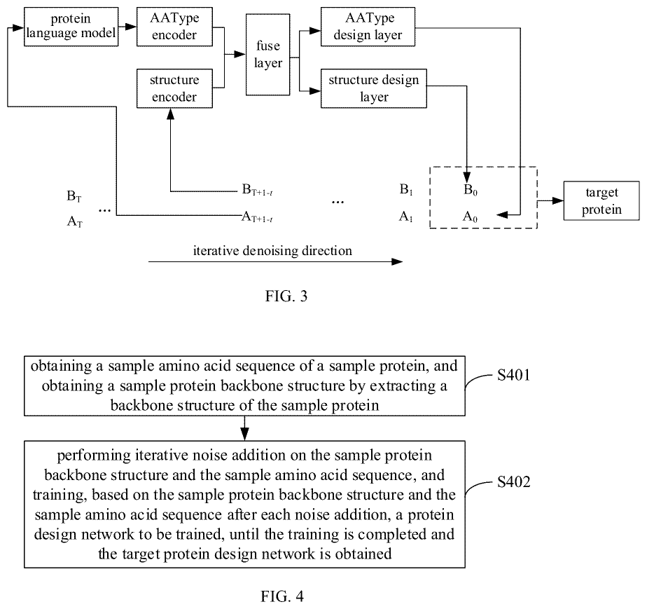

As an exemplary illustration, a diagram of the target protein design network is shown in FIG. 3, which includes a protein language model, an AAType encoder, a structure encoder, a fuse layer, an AAType design layer, and a structure design layer.

For the t^{th} denoising, the residue sequence A_{T+1-t} is input into the protein language model to extract the coevolution information in the residue sequence A_{T+1-t}, and the coevolution information is input into the AAType encoder to obtain the first encoded information. The first protein backbone structure B_{T+1-t} is input into the structure encoder to obtain the second encoded information. Then, the first encoded information and the second encoded information are input into a fuse layer, which fuses the first encoded information and the second encoded information to obtain fused encoded information.

Furthermore, the fused encoded information is input into the AAType design layer, which performs a residue type design on the residue sequence A_{T+1-t} based on the fused encoded information to obtain the residue sequence A_{T-t}. At the same time, the fused encoded information is input into the structure design layer, which performs a structure design on the first protein backbone structure B_{T+1-t} based on the fused encoded information to obtain the first protein backbone structure B_{T-t}. It is continued to denoise the base sequence A_{T-t} and the first protein backbone structure B_{T-t} until the target amino acid sequence A₀ and the second protein backbone structure B₀ are obtained.

Furthermore, the second protein backbone structure can be complemented to obtain a complete target protein. Optionally, a protein side-chain can be obtained according to the target amino acid sequence, and an atomic structure of the second protein backbone structure can be complemented based on the protein side-chain to obtain a final target protein.

Optionally, the protein side-chain of the target amino acid sequence can be obtained based on a side-chain recovery tool.

According to the method for information processing provided by the embodiment of the present disclosure, the residue sequence A_{T} and the first protein backbone structure B_{T} are obtained, the coevolution information of the residue sequence A_{T} is obtained, the coevolution information and the first protein backbone structure B_{T} are encoded respectively, iterative denoising is performed based on the encoded information, to achieve the residue recovery of the amino acid sequence and the prediction of the protein backbone structure, and the target amino acid sequence and the second protein backbone structure are finally obtained. The coevolution information can reveal the interactions between amino acid residues. Combining the coevolution information when denoising helps retain key interactions during the denoising process, thereby improving the accuracy of predicting the amino acid sequence and the protein backbone structure, and enhancing the stability of the protein structure, thereby improving the effects of the protein design. The embodiment of the disclosure introduces a protein language model to obtain the coevolution information of the residue sequence, which can improve the matching degree between the sequence and the structure, thereby improving the denoising effect of the sequence.

Based on the above embodiments, the present disclosure can explain the training process of the target protein design network. As shown in FIG. 4, the training process of the target protein design network may include the following steps at S401-S402.

S401, a sample amino acid sequence of a sample protein are obtained, and a sample protein backbone structure is obtained by extracting a backbone structure of the sample protein.

In some implementations, the sample protein may be obtained from a protein database, and the sample amino acid sequence of the sample protein may be determined based on gene sequencing and translation technology, and a structure analysis may be then performed on the sample protein to extract the sample protein backbone structure.

S402, iterative noise addition on the sample protein backbone structure and the sample amino acid sequence is performed, and a protein design network to be trained is trained based on the sample protein backbone structure and the sample amino acid sequence after each noise addition, until the training is completed and the target protein design network is obtained.

In some implementations, a structural noise and a discrete sequence noise can be used respectively to perform iterative noise addition to the sample protein backbone structure and the sample amino acid sequence, which can accurately simulate a noise mode in real data, and thus improve the accuracy of a reduced result. The structural noise includes a Gaussian noise and a SO3 spatial noise. The discrete sequence noise includes a polynomial diffusion noise and a random mask noise.

That is to say, starting from a first noise addition, for a t^{th} noise addition, a sample amino acid sequence after the t^{th} noise addition and a sample protein backbone structure after the t^{th} noise addition are obtained by performing noise addition processing on a sample amino acid sequence after a (t−1)^{th} noise addition and a sample protein backbone structure after the (t−1)^{th} noise addition respectively.

Optionally, noise addition processing on the sample amino acid sequence may be performed by a residue mask to increase the diversity of the sample amino acid sequence. The candidate residue type is obtained, and a residue mask on the sample amino acid sequence after the (t−1)^{th} noise addition is performed based on the candidate residue type, so as to obtain the sample amino acid sequence after the t^{th} noise addition.

Optionally, a residue type may be randomly selected from a residue type library as a candidate residue type. Optionally, a new residue type is constructed based on a discrete sequence noise, as the candidate residue type, which effectively simulates random errors in real biological data, and thus enhances a design effect of the protein design network.

In some implementations, the protein structure can be perturbed to achieve noise processing on the sample protein backbone structure, which can simulate real scenarios and provide highly flexible sample data. The sample protein backbone structure after the t^{th} noise addition is obtained by perturbing the sample protein backbone structure after the (t−1)^{th} noise addition based on a structural noise.

Optionally, a Gaussian noise can be superimposed on residue coordinates in the sample protein backbone structure after the (t−1)^{th} noise addition, and the sample protein backbone structure after the t^{th} noise addition is obtained by perturbing a backbone rotation direction based on a SO3 spatial noise.

In some implementations, for the sample protein backbone structure and the sample amino acid sequence after each noise addition, a protein design network to be trained can reduce the sample protein backbone structure and sample amino acid sequence after the current noise addition, and it is determined whether a training end condition is met based on a reduced result. If the condition is met, the target protein design network is obtained.

Optionally, the training end condition may be that the number of training times for the protein design network reaches a set number of training times, and the training end condition may also be that the accuracy of the protein design network reaches a set value.

In some implementations, the sample protein backbone structure and the sample amino acid sequence after noise addition are inputted into a protein design network. Then, a reduced sample amino acid sequence and a reduced sample protein backbone structure are outputted by performing reduction based on the sample amino acid sequence and the sample protein backbone structure via the protein design network. A loss function of the protein design network is determined based on the reduced sample amino acid sequence and the reduced sample protein backbone structure, as well as the sample amino acid sequence and the sample protein backbone structure. Then, based on the loss function, model parameters of the protein design network are adjusted to enhance a design effect of the protein design network.

According to the method for information processing provided by the embodiment of the present disclosure, the sample amino acid sequence and the sample protein backbone structure of the sample protein are obtained, and using different noise addition ways, iterative noise addition on the sample amino acid sequence and the sample protein backbone structure are performed respectively, and the protein design network to be trained is trained based on the sample amino acid sequence and the sample protein backbone structure after noise addition, until the target protein design network is obtained, which can effectively improve the generalization ability of the protein design network, promote the protein design network to learn more detailed information, and enhance a design effect of the target protein design network.

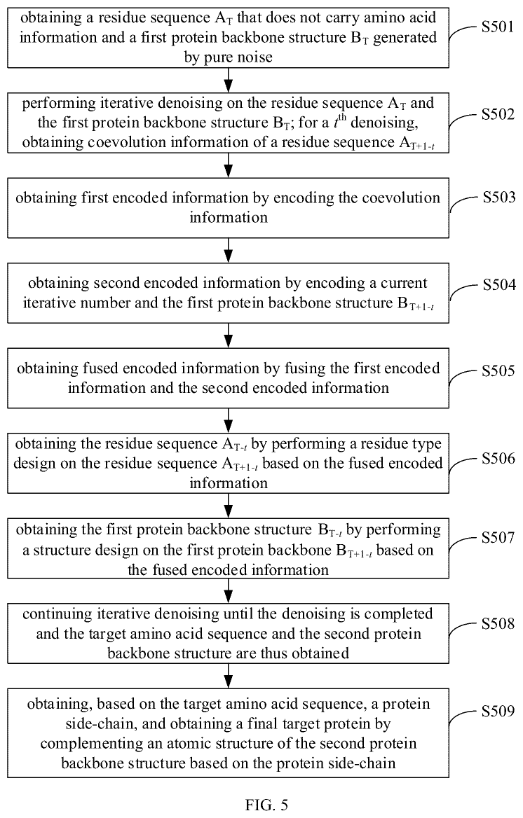

FIG. 5 is a flowchart of another method for information processing according to an embodiment of the disclosure.

As shown in FIG. 5, the method for information processing may include the following steps at S501-S509.

At S501, a residue sequence A_{T} that does not carry amino acid information and a first protein backbone structure B_{T} generated by pure noise are obtained.

At S502, iterative denoising on the residue sequence A_{T} and the first protein backbone structure B_{T} is performed; for the t^{th} denoising, coevolution information of a residue sequence A_{T+1-t} is obtained.

At S503, first encoded information is obtained by encoding the coevolution information.

At S504, second encoded information is obtained by encoding a current iterative number and the first protein backbone structure B_{T+1-t}.

At S505, fused encoded information is obtained by fusing the first encoded information and the second encoded information.

At S506, the residue sequence A_{T-t} is obtained by performing a residue type design on the residue sequence A_{T+1-t} based on the fused encoded information.

At S507, the first protein backbone structure B_{T-t} is obtained by performing a structure design on the first protein backbone B_{T+1-t} based on the fused encoded information.

At S508, iterative denoising is continued to perform until the denoising is completed and the target amino acid sequence and the second protein backbone structure are thus obtained.

At S509, a protein side-chain is obtained based on the target amino acid sequence, and a final target protein is obtained by complementing an atomic structure of the second protein backbone structure based on the protein side-chain.

The relevant contents of steps S501-S509 can be referred to the above steps in the embodiments, which will not be repeated here.

According to the method for information processing provided by the embodiment of the present disclosure, the residue sequence A_{T} and the first protein backbone structure B_{T} are obtained, the coevolution information of the residue sequence A_{T} is obtained, the coevolution information and the first protein backbone structure B_{T} are encoded respectively, iterative denoising is performed based on the encoded information, to achieve the residue recovery of the amino acid sequence and the prediction of the protein backbone structure, and the target amino acid sequence and the second protein backbone structure are finally obtained. The coevolution information can reveal the interactions between amino acid residues. Combining the coevolution information when denoising helps retain key interactions during the denoising process, thereby improving the accuracy of predicting the amino acid sequence and the protein backbone structure, and enhancing the stability of the protein structure, thereby improving the effects of the protein design. The embodiment of the disclosure introduces a protein language model to obtain the coevolution information of the residue sequence, which can improve the matching degree between the sequence and the structure, thereby improving the denoising effect of the sequence.

Corresponding to the method for information processing provided in the above-mentioned embodiments, an embodiment of the present disclosure further provides an apparatus for information processing. Since the apparatus for information processing provided in the embodiment of the present disclosure corresponds to the method for information processing provided in the above-mentioned embodiments, the implementation of the above-mentioned method for information processing are also applicable to those of the apparatus for information processing provided in the embodiment of the present disclosure, which will not be described in detail below.

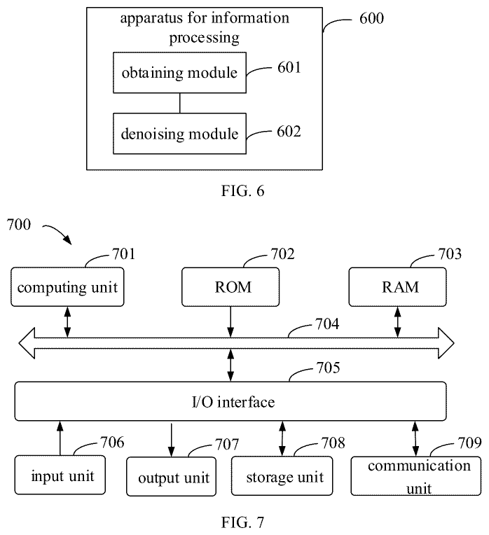

FIG. 6 is a structural diagram of an apparatus for information processing according to an embodiment of the disclosure.

As shown in FIG. 6, the apparatus for information processing 600 according to the embodiment of the present disclosure includes an obtaining module 601 and a denoising module 602.

The obtaining module 601 is configured to obtain a residue sequence A_{T} that does not carry amino acid information and a first protein backbone structure B_{T} generated by pure noise.

The denoising module 602 is configured to perform iterative denoising on the residue sequence A_{T} and the first protein backbone structure B_{T}; for a t^{th} denoising, obtain coevolution information of a residue sequence A_{T+1-t}, and obtain, based on the coevolution information and a first protein backbone structure B_{T+1-t}, a residue sequence A_{T-t} and a first protein backbone structure B_{T-t} after the t^{th} denoising, until the denoising is completed and a target amino acid sequence and a second protein backbone structure are obtained, where t is a positive integer, and 1≤t≤T, and T is a number of denoising times.

In an embodiment of the present disclosure, the denoising module 602 is further configured to: obtain first encoded information by encoding the coevolution information; obtain second encoded information by encoding a current iterative number and the first protein backbone structure B_{T+1-t}; and obtain, based on the first encoded information and the second encoded information, the residue sequence A_{T-t} and the first protein backbone structure B_{T-t} after the t^{th} denoising.

In an embodiment of the present disclosure, the denoising module 602 is further configured to: obtain fused encoded information by fusing the first encoded information and the second encoded information; obtain the residue sequence A_{T-t} by performing a residue type design on the residue sequence A_{T+1-t} based on the fused encoded information; and obtain the first protein backbone structure B_{T-t} by performing a structure design on the first protein backbone B_{T+1-t} based on the fused encoded information.

In an embodiment of the present disclosure, the denoising module 602 is further configured to: input the residue sequence A_{T} and the first protein backbone structure B_{T} into a pre-trained target protein design network, and output the second protein backbone structure and the target amino acid sequence by performing iterative denoising on the residue sequence A_{T} and the first protein backbone structure B_{T} via the target protein design network.

In an embodiment of the present disclosure, the denoising module 602 is further configured to: for the t^{th} denoising, extract the coevolution information of the residue sequence A_{T+1-t} with a protein language model in the target protein design network and obtain, based on the coevolution information and the first protein backbone structure B_{T+1-t}, the residue sequence A_{T-t} and the first protein backbone structure B_{T-t} after the t^{th} denoising.

In an embodiment of the present disclosure, the denoising module 602 is further configured to: obtain, based on the target amino acid sequence, a protein side-chain, and obtain a final target protein by complementing an atomic structure of the second protein backbone structure based on the protein side-chain.

In an embodiment of the present disclosure, the denoising module 602 is further configured to: obtain a sample amino acid sequence of a sample protein, and obtain a sample protein backbone structure by extracting a backbone structure of the sample protein; and perform iterative noise addition on the sample protein backbone structure and the sample amino acid sequence, and train, based on the sample protein backbone structure and the sample amino acid sequence after each noise addition, a protein design network to be trained, until the training is completed and the target protein design network is obtained.

In an embodiment of the present disclosure, the denoising module 602 is further configured to: input the sample protein backbone structure and the sample amino acid sequence after noise addition into the protein design network, and obtain a reduced sample amino acid sequence and a reduced sample protein backbone structure by performing reduction based on the sample amino acid sequence and the sample protein backbone structure via the protein design network; determine, based on the reduced sample amino acid sequence and the reduced sample protein backbone structure, and the sample amino acid sequence and the sample protein backbone structure, a loss function of the protein design network; and adjust, based on the loss function, model parameters of the protein design network.

In an embodiment of the present disclosure, the denoising module 602 is further configured to: starting from a first noise addition, for a t^{th} noise addition, obtain a sample amino acid sequence after the t^{th} noise addition and a sample protein backbone structure after the t^{th} noise addition by performing noise addition processing on a sample amino acid sequence after a (t−1)^{th} noise addition and a sample protein backbone structure after the (t−1)^{th} noise addition.

In an embodiment of the present disclosure, the denoising module 602 is further configured to: obtain a candidate residue type; and obtain the sample amino acid sequence after the t^{th} noise addition by performing a residue mask on the sample amino acid sequence after the (t−1)^{th} noise addition based on the candidate residue type.

In an embodiment of the present disclosure, the denoising module 602 is further configured to: randomly select a residue type from a residue type library as the candidate residue type; or construct, based on a discrete sequence noise, a new residue type as the candidate residue type.

In an embodiment of the present disclosure, the denoising module 602 is further configured to: obtain the sample protein backbone structure after the t^{th} noise addition by perturbing the sample protein backbone structure after the (t−1)^{th} noise addition based on a structural noise.

In an embodiment of the present disclosure, the denoising module 602 is further configured to: superimpose a Gaussian noise on residue coordinates in the sample protein backbone structure after the (t−1)^{th} noise addition, and obtain the sample protein backbone structure after the t^{th} noise addition by perturbing a backbone rotation direction based on a SO3 spatial noise.

According to the apparatus for information processing provided in the embodiment of the present disclosure, the residue sequence A_{T} and the first protein backbone structure B_{T} are obtained, the coevolution information of the residue sequence A_{T} is obtained, iterative denoising is performed based on the coevolution information and the first protein backbone structure B_{T}, to achieve the residue recovery of the amino acid sequence and the prediction of the protein backbone structure, and the target amino acid sequence and the second protein backbone structure are finally obtained. The coevolution information can reveal the interactions between amino acid residues. Combining the coevolution information when denoising helps retain key interactions during the denoising process, thereby improving the accuracy of predicting an amino acid sequence and a protein backbone structure, and enhancing the stability of a protein structure, thereby improving the effects of a protein design.

In the technical solution of the disclosure, the acquisition, storage, and application, of personal information of users are all in compliance with relevant laws and regulations, and do not violate public order and morals.

According to embodiments of the disclosure, it also provides an electronic device, a readable storage medium, and a computer program product.

Referring to FIG. 7, it is a block diagram illustrating an electronic device 700 according to an embodiment of the disclosure. The electronic device is intended to represent various types of digital computers, such as laptop computers, desktop computers, workstations, personal digital assistants, servers, blade servers, mainframe computers, and other suitable computers. The electronic device may also represent various types of mobile devices, such as personal digital assistants, cellular phones, smart phones, wearable devices, and other similar computing devices. The components shown herein, their connections and relations, and their functions are merely examples, which are not intended to limit the implementations of the disclosure described and/or required herein.

As shown in FIG. 5, the device 700 includes a computing unit 701, configured to execute various appropriate actions and processes according to computer programs/instructions stored in a read-only memory (ROM) 702 or computer programs loaded from a storage unit 708 to a random access memory (RAM) 703. In the RAM 703, various programs and data required for the device 700 may be stored. The computing unit 701, the ROM 702 and the RAM 703 may be connected with each other by a bus 704. An input/output (I/O) interface 705 is also connected to the bus 704.

The plurality of components in the device 700 are connected to the I/O interface 705, which include: an input unit 506, for example, a keyboard, a mouse; an output unit 707, for example, various types of displays, speakers; a storage unit 708, for example, a magnetic disk, an optical disk; and a communication unit 709, for example, a network card, a modem, a wireless transceiver. The communication unit 709 allows the device 700 to exchange information/data through a computer network such as Internet and/or various types of telecommunication networks with other devices.

The computing unit 701 may be various types of general and/or dedicated processing components with processing and computing abilities. Some examples of a computing unit 701 include but not limited to a central processing unit (CPU), a graphics processing unit (GPU), various dedicated artificial intelligence (AI) computing chips, various computing units on which a machine learning model algorithm is running, a digital signal processor (DSP), and any appropriate processor, controller, microcontroller, etc. The computing unit 701 executes various methods and processes as described above, for example, a method for information processing. For example, in some embodiments, the method for information processing may be further implemented as a computer software program, which is tangibly contained in a machine readable medium, such as the storage unit 708. In some embodiments, a part or all of the computer program may be loaded and/or installed on the device 700 via the ROM 702 and/or the communication unit 709. When the computer program is loaded on the RAM 703 and executed by the computing unit 701, one or more steps in the method for information processing may be performed as described above. Optionally, in other embodiments, the computing unit 701 may be configured to the method for information processing in other appropriate ways (for example, by virtue of a firmware).

Various implementations of the systems and techniques described above may be implemented by a digital electronic circuit system, an integrated circuit system, Field Programmable Gate Arrays (FPGAs), Application Specific Integrated Circuits (ASICs), Application Specific Standard Products (ASSPs), System on Chip (SOCs), Load programmable logic devices (CPLDs), computer hardware, firmware, software, and/or a combination thereof. These various embodiments may be implemented in one or more computer programs/instructions, the one or more computer programs/instructions may be executed and/or interpreted on a programmable system including at least one programmable processor, which may be a dedicated or general programmable processor for receiving data and instructions from the storage system, at least one input device and at least one output device, and transmitting the data and instructions to the storage system, the at least one input device and the at least one output device.

The program code configured to implement the method of the disclosure may be written in any combination of one or more programming languages. These program codes may be provided for the processors or controllers of general-purpose computers, dedicated computers, or other programmable data processing devices, so that the program codes, when executed by the processors or controllers, enable the functions/operations specified in the flowchart and/or block diagram to be implemented. The program code may be executed entirely on the machine, partly executed on the machine, partly executed on the machine and partly executed on the remote machine as an independent software package, or entirely executed on the remote machine or server.

In the context of the disclosure, a machine-readable medium may be a tangible medium that may contain or store a program for use by or in combination with an instruction execution system, apparatus, or device. The machine-readable medium may be a machine-readable signal medium or a machine-readable storage medium. A machine-readable medium may include, but is not limited to, an electronic, magnetic, optical, electromagnetic, infrared, or semiconductor system, apparatus, or device, or any suitable combination of the foregoing. More specific examples of machine-readable storage media include electrical connections based on one or more wires, portable computer disks, hard disks, RAMs, ROMs, Electrically Programmable Read-Only-Memory (EPROM), fiber optics, Compact Disc Read-Only Memories (CD-ROM), optical storage devices, magnetic storage devices, or any suitable combination of the foregoing.

In order to provide interaction with a user, the systems and techniques described herein may be implemented on a computer having a display device (e.g., a Cathode Ray Tube (CRT) or a Liquid Crystal Display (LCD) monitor for displaying information to a user); and a keyboard and pointing device (such as a mouse or trackball) through which the user can provide input to the computer. Other kinds of devices may also be used to provide interaction with the user. For example, the feedback provided to the user may be any form of sensory feedback (e.g., visual feedback, auditory feedback, or haptic feedback), and the input from the user may be received in any form (including acoustic input, voice input, or tactile input).

The systems and technologies described herein can be implemented in a computing system that includes background components (for example, a data server), or a computing system that includes middleware components (for example, an application server), or a computing system that includes front-end components (for example, a user computer with a graphical user interface or a web browser, through which the user can interact with the implementation of the systems and technologies described herein), or include such background components, intermediate computing components, or any combination of front-end components. The components of the system may be interconnected by any form or medium of digital data communication (e.g., a communication network). Examples of communication networks include: a Local Area Network (LAN), a Wide Area Network (WAN), the Internet and a blockchain network.

The computer system may include a client and a server. The client and server are generally remote from each other and interacting through a communication network. The client-server relation is generated by computer programs/instructions running on the respective computers and having a client-server relation with each other. The server may be a cloud server, a server of a distributed system, or a server combined with a block-chain.

It should be understood that the various forms of processes shown above can be used to reorder, add or delete steps. For example, the steps described in the disclosure could be performed in parallel, sequentially, or in a different order, as long as the desired result of the technical solution disclosed in the disclosure is achieved, which is not limited herein.

The above specific embodiments do not constitute a limitation on the protection scope of the disclosure. Those skilled in the art should understand that various modifications, combinations, sub-combinations and substitutions can be made according to design requirements and other factors. Any modification, equivalent replacement and improvement made within the principle of the disclosure shall be included in the protection scope of the disclosure.

What is claimed is:

1. A method for information processing, performed by an electronic device, comprising:
obtaining a residue sequence A_{T} that does not carry amino acid information and a first protein backbone structure B_{T} generated by pure noise; and
performing iterative denoising on the residue sequence A_{T} and the first protein backbone structure B_{T}; for a t^{th} denoising, obtaining coevolution information of a residue sequence A_{T+1-t}, and obtaining, based on the coevolution information and a first protein backbone structure B_{T+1-t}, a residue sequence A_{T-t} and a first protein backbone structure B_{T-t} after the t^{th} denoising, until the denoising is completed and a target amino acid sequence and a second protein backbone structure are obtained, wherein t is a positive integer, and 1≤t≤T, and T is a number of denoising times.

2. The method according to claim 1, wherein obtaining the residue sequence A_{T-t} and the first protein backbone structure B_{T-t} after the t^{th} denoising, comprises:
obtaining first encoded information by encoding the coevolution information;
obtaining second encoded information by encoding a current iterative number and the first protein backbone structure B_{T+1-t}; and
obtaining, based on the first encoded information and the second encoded information, the residue sequence A_{T-t} and the first protein backbone structure B_{T-t} after the t^{th} denoising.

3. The method according to claim 2, wherein obtaining, based on the first encoded information and the second encoded information, the residue sequence A_{T-t} and the first protein backbone structure B_{T-t} after the t^{th} denoising, comprises:
obtaining fused encoded information by fusing the first encoded information and the second encoded information;
obtaining the residue sequence A_{T-t} by performing a residue type design on the residue sequence A_{T+1-t} based on the fused encoded information; and
obtaining the first protein backbone structure B_{T-t} by performing a structure design on the first protein backbone B_{T+1-t} based on the fused encoded information.

4. The method according to claim 1, further comprising:
inputting the residue sequence A_{T} and the first protein backbone structure B_{T} into a pre-trained target protein design network, and outputting the second protein backbone structure and the target amino acid sequence by performing iterative denoising on the residue sequence A_{T} and the first protein backbone structure B_{T} via the target protein design network.

5. The method according to claim 4, wherein performing iterative denoising on the residue sequence A_{T} and the first protein backbone structure B_{T} via the target protein design network, comprises:
for the t^{th} denoising, extracting the coevolution information of the residue sequence A_{T+1-t} with a protein language model in the target protein design network and obtaining, based on the coevolution information and the first protein backbone structure B_{T+1-t}, the residue sequence A_{T-t} and the first protein backbone structure B_{T-t} after the t^{th} denoising.

6. The method according to claim 1, further comprising:
obtaining, based on the target amino acid sequence, a protein side-chain, and obtaining a final target protein by complementing an atomic structure of the second protein backbone structure based on the protein side-chain.

7. The method according to claim 4, wherein a training process of the target protein design network comprises:
obtaining a sample amino acid sequence of a sample protein, and obtaining a sample protein backbone structure by extracting a backbone structure of the sample protein; and
performing iterative noise addition on the sample protein backbone structure and the sample amino acid sequence, and training, based on the sample protein backbone structure and the sample amino acid sequence after each noise addition, a protein design network to be trained, until the training is completed and the target protein design network is obtained.

8. The method according to claim 7, wherein training the protein design network comprises:
inputting the sample protein backbone structure and the sample amino acid sequence after noise addition into the protein design network, and obtaining a reduced sample amino acid sequence and a reduced sample protein backbone structure by performing reduction based on the sample amino acid sequence and the sample protein backbone structure via the protein design network;
determining, based on the reduced sample amino acid sequence and the reduced sample protein backbone structure, and the sample amino acid sequence and the sample protein backbone structure, a loss function of the protein design network; and
adjusting, based on the loss function, model parameters of the protein design network.

9. The method according to claim 8, further comprising:
starting from a first noise addition, for a t^{th} noise addition, obtaining a sample amino acid sequence after the t^{th} noise addition and a sample protein backbone structure after the t^{th} noise addition by performing noise addition on a sample amino acid sequence after a (t−1)^{th} noise addition and a sample protein backbone structure after the (t−1)^{th} noise addition.

10. The method according to claim 9, further comprising:
obtaining a candidate residue type; and
obtaining the sample amino acid sequence after the t^{th} noise addition by performing a residue mask on the sample amino acid sequence after the (t−1)^{th} noise addition based on the candidate residue type.

11. The method according to claim 10, wherein obtaining the candidate residue type comprises:
randomly selecting a residue type from a residue type library as the candidate residue type; or
constructing, based on a discrete sequence noise, a new residue type as the candidate residue type.

12. The method according to claim 9, further comprising:
obtaining the sample protein backbone structure after the t^{th} noise addition by perturbing the sample protein backbone structure after the (t−1)^{th} noise addition based on a structural noise.

13. The method according to claim 12, wherein obtaining the sample protein backbone structure after the t^{th} noise addition by perturbing the sample protein backbone structure after the (t−1)^{th} noise addition based on the structural noise, comprises:
superimposing a Gaussian noise on residue coordinates in the sample protein backbone structure after the (t−1)^{th} noise addition, and obtaining the sample protein backbone structure after the t^{th} noise addition by perturbing a backbone rotation direction based on a SO3 spatial noise.

14. An electronic device, comprising:
at least one processor; and
a memory communicatively coupled to the at least one processor and storing instructions executable by the at least one processor;
wherein when the instructions are executed by the at least one processor, the at least one processor is configured to:
obtain a residue sequence A_{T} that does not carry amino acid information and a first protein backbone structure B_{T} generated by pure noise; and
perform iterative denoising on the residue sequence A_{T} and the first protein backbone structure B_{T}; for a t^{th} denoising, obtain coevolution information of a residue sequence A_{T+1-t}, and obtain, based on the coevolution information and a first protein backbone structure B_{T+1-t}, a residue sequence A_{T-t} and a first protein backbone structure B_{T-t} after the t^{th} denoising, until the denoising is completed and a target amino acid sequence and a second protein backbone structure are obtained, wherein t is a positive integer, and 1≤t≤T, and T is a number of denoising times.

15. The electronic device according to claim 14, wherein the at least one processor is further configured to:
obtain first encoded information by encoding the coevolution information;
obtain second encoded information by encoding a current iterative number and the first protein backbone structure B_{T+1-t}; and
obtain, based on the first encoded information and the second encoded information, the residue sequence A_{T-t} and the first protein backbone structure B_{T-t} after the t^{th} denoising.

16. The electronic device according to claim 15, wherein the at least one processor is further configured to:
obtain fused encoded information by fusing the first encoded information and the second encoded information;
obtain the residue sequence A_{T-t} by performing a residue type design on the residue sequence A_{T+1-t} based on the fused encoded information; and
obtain the first protein backbone structure B_{T-t} by performing a structure design on the first protein backbone B_{T+1-t} based on the fused encoded information.

17. The electronic device according to claim 14, wherein the at least one processor is further configured to:
input the residue sequence A_{T} and the first protein backbone structure B_{T} into a pre-trained target protein design network, and output the second protein backbone structure and the target amino acid sequence by performing iterative denoising on the residue sequence A_{T} and the first protein backbone structure B_{T} via the target protein design network.

18. The electronic device according to claim 17, wherein the at least one processor is further configured to:
for the t^{th} denoising, extract the coevolution information of the residue sequence A_{T+1-t} with a protein language model in the target protein design network and obtain, based on the coevolution information and the first protein backbone structure B_{T+1-t}, the residue sequence A_{T-t} and the first protein backbone structure B_{T-t} after the I^{th} denoising.

19. The electronic device according to claim 14, wherein the at least one processor is further configured to:
obtain, based on the target amino acid sequence, a protein side-chain, and obtain a final target protein by complementing an atomic structure of the second protein backbone structure based on the protein side-chain.

20. A non-transitory computer readable storage medium, storing computer instructions, wherein the computer instructions are caused to enable a computer to perform a method for information processing, the method comprising:
obtaining a residue sequence A_{T} that does not carry amino acid information and a first protein backbone structure B_{T} generated by pure noise; and
performing iterative denoising on the residue sequence A_{T} and the first protein backbone structure B_{T}; for a t^{th} denoising, obtaining coevolution information of a residue sequence A_{T+1-t}, and obtaining, based on the coevolution information and a first protein backbone structure B_{T+1-t}, a residue sequence A_{T-t} and a first protein backbone structure B_{T-t} after the t^{th} denoising, until the denoising is completed and a target amino acid sequence and a second protein backbone structure are obtained, wherein t is a positive integer, and 1≤t≤T, and T is a number of denoising times.
